SEQUENCE LISTING (5 sequences)

SEQ ID NO: 1 - DNA, 20 bp, SARS-CoV-2
gccaatgcgcgacattccga

SEQ ID NO: 2 - DNA, 20 bp, SARS-CoV-2
agaacgctgaagcgctgggg

SEQ ID NO: 3 - DNA, 20 bp, SARS-CoV-2
tacactcgatcgtactccgc

SEQ ID NO: 4 - DNA, 20 bp, SARS-CoV-2
cgtggcctcggtgaaaatgt

SEQ ID NO: 5 - DNA, 20 bp, SARS-CoV-2
ctccctagcattgttcactg

(19) United States
(12) Patent Application Publication
(10) Pub. No.: US 2025/0277255 A1
(43) Pub. Date: Sep. 4, 2025

(54) A LARGE CAPACITY CLINICAL ASSAY METHOD

(71) Applicants: Timo RAPAKKO, Saratoga, CA (US); Jorma VIRTANEN, Massillon, OH (US)

(72) Inventors: Timo RAPAKKO, Saratoga, CA (US); Jorma VIRTANEN, Massillon, OH (US)

(21) Appl. No.: 18/562,532

(22) PCT Filed: May 19, 2022
(86) PCT No.: PCT/US2022/029948

§ 371 Date: Nov. 20, 2023

Related U.S. Application Data
(60) Provisional application No. 63/258,671, filed on May 21, 2021.

(51) Int. Cl. C12Q 1/6825; C12Q 1/70

(52) U.S. Cl. CPC C12Q 1/6825; C12Q 1/701

(57) ABSTRACT

Described herein are various methods and embodiments of multibead detection of an analyte, such as a polynucleotide or a protein. The method is characterized by the use of multiple probes in the capture bead, or surface, and also in the reporter bead. Thus, even weak binding may lead to efficient overall binding.

### TECHNICAL FIELD

The subject matter described herein relates to various methods and embodiments of a clinical chemistry assays of oligonucleotides and antigens. More specifically, optical methods are used to detect a double bead complex after filtration.

### REFERENCE TO AN ELECTRONIC SEQUENCE LISTING

The contents of the electronic sequence listing (43689_50005_SeqListing.txt; Size: 1020 bytes; and Date of Creation: May 10, 2024) is hereby incorporated by reference in its entirety.

### BACKGROUND AND PRIOR ART

The importance of clinical chemical testing has become more important during SARS-COV-2 pandemic. There are numerous testing methods for viruses, and other pathogens. Most importantly PCR method has been widely used for the detection of polynucleotides. In principle, PCR has an ultimate sensitivity, because even one molecule may multiplied million, or billionfold, or even more. Then detection with standard methods is possible, including gel electrophoresis, and more recently using specific dyes that turn fluorescent, when double stranded DNA is present.

Pathogens may also be detected by immunoassays. Viruses have protein capside surrounding the genetic material. Antibodies against these proteins may be fabricated. Similarly, bacterial membranes contain proteins. For analytical purposes bacteria or viruses may be broken into molecular components. Two antibodies may bind same protein at the same time, or alternatively one antibody may bind one protein on an intact bacterium or virus, another antibody may bind another protein on the same bacterium or virus. One antibody may be on a solid surface, such as on a bottom of a well plate, or may be immobilized at later stages of an assay. When a sample containing an antigen is placed into a well, antigen will be immobilized, and can still bind another antibody that carries a reporter agent that may be optically detectable molecule, a polymeric bead containing hundreds of optically detectable molecules, radioactive element, or enzyme that is able to generate optically detectable signal from a substrate that gets stronger over time. The latter method is called ELISA (Enzyme Linked Immunoabsorbent Assay).

Dual Bead method is analogous to these methods in the sense that a target will bind with capture bead, and reporter bead (K. Mullis, B. Phan, and J. Virtanen, Dual Bead Assays Using Cleavable Spacers and/or Ligation to Improve Specificity and Sensitivity Including Related Methods and Apparatus, WO 02/073605 A3, 2002, US20050069923, provided as a reference in its entirety). In dual bead method (more correctly double bead method) it is important to separate free reporter beads from the bound reporter beads before optical measurement. In the method of Mullis, et al. this was achieved either with magnetic field or centrifugal force. For this reason the capture beads were paramagnetic beads that are also heavier than conventional plastic beads.

The present method is a double bead method, in which separation is based on filtration. Only requirement for the capture beads is that they are bigger than the reporter beads, and also bigger than the pore size of the filter.

### SUMMARY

The following clauses summarize the present invention:

Clause 1. A method for the detection of an analyte in a sample by
a. adding capture beads, and
b. reporter beads, and
c. incubating the mixture, and
d. filtering the mixture through a filter that has pore size that is at least 20% bigger than the diameter of the reporter beads, but at least 50% smaller than the diameter of the capture beads, and
e. using an optical reader to measure the amount of the reporter beads retained by the filter.

Clause 2. The method of Clause 1, in which the analyte is an oligonucleotide, and the capture, and reporter beads are conjugated with complementary oligonucleotide probes.

Clause 3. The method of Clause 1, in which the analyte is an antigen, and the capture, and reporter beads are conjugated with antibodies that are specific for the said antigen.

Clause 4. The method of Clause 1, in which the reporter beads contain fluorophores, and the optical reader measures fluorescent light.

Clause 5. The method of Clause 4, in which the optical reader is a digital camera, and the color information if provided for each pixel.

Clause 6. The method of Clause 5, in which the digital information is used to calculate the number of analyte molecules.

Clause 7. The method of claim 1, in which two or more different reporter beads are used to detect the same analyte so that several different colors are detected in a deliberately chosen ratio, when an analyte is present.

Clause 8. The method of Clause 1, in which the said filter is a filter well plate.

Clause 9. The method of Clause 8, in which the said filter well plate is black.

Clause 10. The method of Clause 8, in which at least part of the filter plate is functionalized with biotin, or avidin.

Clause 11. The method of Clause 8, in which the said filter well plater is washed in an ultrasonic bath before the optical measurement.

Clause 12. The method of Clause 8, in which the bottom side of the said filter plate is optically measured.

### FIGURE CAPTIONS

FIG. 1. A. A 96-Well plate. B. A bottom of one filter well containing a large number of pores.

FIG. 2. A. Capture bead, and reporter bead conjugated with binding molecules, and the target.
B. The formation of a double bead complex

FIG. 3. A. Schematics of free capture, and reporter beads, and double bead complex (same as FIG. 2 B). B. A small part of a filter well bottom. C. After filtration all capture beads are retained by the filter, and only those report reads that are bound with capture beads will be retained, while free reporter beads are in the filtrate.

FIG. 4. Depiction of various beads, and complexes that can be deposited on a filter.

FIG. 5. Schematic depiction of optical excitation of the reporter beads. A. From above, B. from below.

FIG. 6. A. Schematics of a filter well plate that maintains the optical focus, B. after turning upside down.

FIG. 7. Experimental detection of the red reporter beads. Red has been changed black, and the black background white for clarity.

FIG. 8. A schematic depiction of electrophoretic implementation of the present invention.

### DEFINITIONS

Analyte is an oligo-, or polynucleotide, or antigen of interest in a sample.

Antigen is a molecule that stimulates the formation of specific antibodies, when the antigen is introduced in vivo into living tissue, notably, antibodies can also be antigens.

Capture bead is a bead made of a solid material, such as plastic, and specific binding molecules, such as oligonucleotides, or antibodies have been conjugated with the bead. For the purposes of this invention the capture bead should be as transparent as possible, and bigger than the reporter bead. In some applications a macroscopic capture surface is used instead of a bead.

Reporter bead has some property that allows its easy optical detection, such as fluorescence. Reporter beads are also conjugated with specific binding molecules that are able to bind the same analyte or target as capture beads

Double bead is a complex that is formed, when capture and reporter bead bind the same analyte molecule.

Multibead complex is formed when one capture bead binds several reporter beads. The binding is mediated by an analyte, or several analyte molecules. Reporter beads may be same kind or different.

Digital camera. Camera that has an array of optical sensors, and is able to store the optical information for each pixel. Modern digital cameras also measure, and store color information of each pixel. Typically, the intensity of blue, green, and red light at each pixel is measured, and each pixel may contain 16 or more color sensitive light detectors.

### DETAILED DESCRIPTION OF THE INVENTION

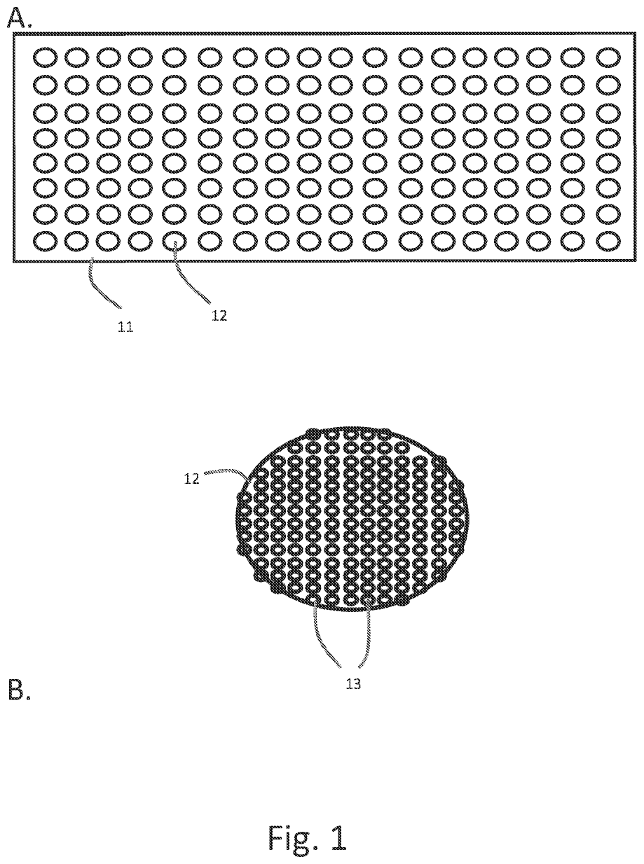

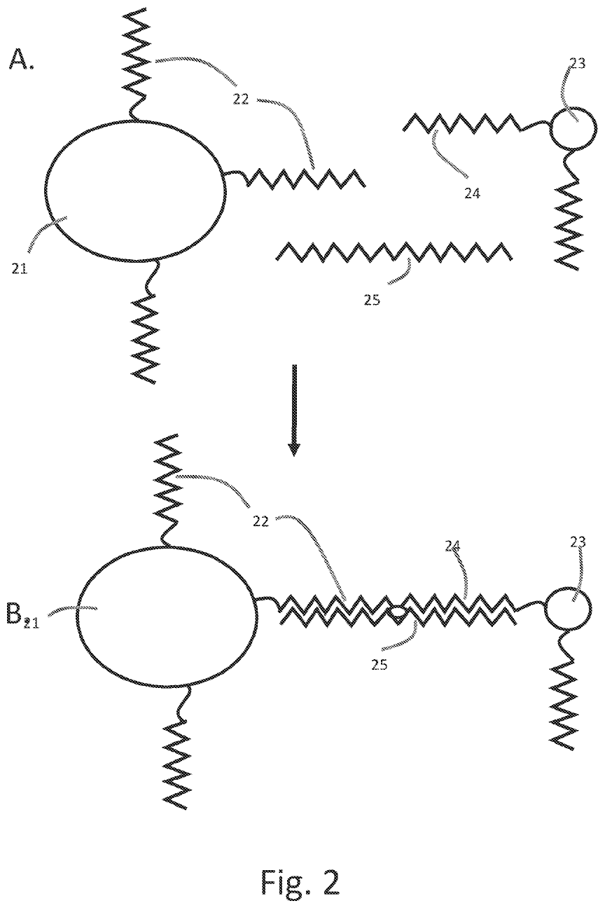

This invention is based on the formation of double bead or multibead complexes between a capture bead, and reporter beads. These complexes (FIG. 2B) will be formed only, if a proper target molecule 25, or analyte is present in the sample (FIG. 2). Thus, the formation of these complexes can be used to measure the amount of a target molecules. In order to optically measure the amount of bound reporter beads 26, the free reporter beads 23 must be separated from the bound ones. In the method of the present invention this separation is performed by filtration. For this purpose the capture beads 21 should be so big that they, and double, and multibeads are retained on the filter, while free reported beads go through the filter to the filtrate. Filtrate is discarded, and the filter plate is red by optical reader. Filtration and optical reading may be done in a large scale using well plate technology. In FIG. 1 is depicted a typical 96-well plate 11. Wells may have continuous smooth plastic bottom, or porous bottom 12 with well defined pores 13 (FIG. 1 B).

Reporter beads may be made of the same materials than the capture beads, and also functionalized analogously. Reporter beads should contain fluorophores, or other molecules that may be detected easily by optical methods. Fluorophores may be inside, and/or on the surface of the bead. Very large number of fluorophores are known. Most are organic dyes. Also transition metal chelates may be used. Recently, quantum dots have been commercially available. Quantum dots have the advantage that excitation, and emission wavelengths may be widely different. This helps to minimize background radiation. Some rare earth, such as europium chelates have same property, excitation is under 400 nm, while emission is over 600 nm, more than 200 nm difference that is unheard of traditional molecular fluorophores.

FIG. 2 depicts the formation of the double bead complex. In this example the capture bead 21 has been coated with oligonucleotide probes 22 that are specific for the target 25. In this schematic figure only three probes are shown, but the capture bead can have hundreds or even thousands of probes that may be identical or different. Use of multiple different probes that are specific for same target leads to a stronger overall binding. All probes are designed to bind a specific target that may be a DNA or RNA fragment of pathogenic genome, such as viral or bacterial genome. In this example the probes are oligonucleotides, but they may be peptide nucleic acids, or other nucleotide analogs. Reporter beads 23 have also been coated with probes 24 that are complementary to the target, but at different segment than the probes on the capture bead.

Especially, polynucleotides may bind several oligonucleotide probes. For example, capture bead 91 (FIG. 9) may have three or more probes that all bind with same polynucleotide. Similarly, reporter beads 92 may have three or more oligonucleotide probes that bind with same polynucleotide than capture bead probes. Advantageously, capture, and reporter probes bind alternatively, i.e., binding order is capture probe 1, reporter probe 1, capture probe 2, reporter probe 2, capture probe 3, reporter probe 3, etc. (FIG. 9). Capture probe binding sites are denoted 95, while reporter bead binding sited are denoted 94 in FIG. 9. The target polynucleotide 93 has 3′-enf on the left side, and 5′-end on the right side. Capture and reporter bead probe spacers may be in 3′-end, or in 5′-end, although 3′-end is preferred for spacers. This structure is net-like, in which tension is distributed 2-, or 3-dimensionally unlike in a chain, in which the weakest link dictates the strength.

It is possible to use multiple different reporter beads. They might have same fluorophore, but different probes that bind the same target, but at another site. This would amplify the optical signal. It might also be advantageous to use different fluorophores. For example, one reporter bead could emit green light, and the other one red light. In this case the target oligonucleotide that is on the capture bead could bind both equally. Then green, and red emission ratio 1:1 could be observed in the same spot. The reporter beads could also be designed so that for each green bead two red beads will bind. Then the intensity ratio would be 1:2. This would help to differentiate nonspecific binding from specific binding. Nonspecific binding gives most of the time a random ratio of green or red at the same spot, and rarely a given ratio, such as 1:2, or 1:3, etc., which are possible by design. In order to further clarify this aspect of the present invention, the green and red fluorescent reporter beads may be added into the assay mixture, for example, in 2:1 ratio, although the green beads have only one binding site in the target molecule, while the red beads have two binding sites. Non-specific binding happens randomly, and would give average binding ratio 2:1 in any given small area (and also in a larger area). However, specific binding would give the ratio 1:2. More than two different fluorophores may be used in order to improve specificity of the assay. Target oligonucleotides are often so long that they can bind tens of different reporter beads.

Each supramolecular complex gives a certain pixel the color that may be observed. Thus, the total number of supramolecular complexes may be calculated. Each supramolecular complex contains at least one analyte molecule. If it contains several analyte molecules, intensity data may be used to estimate the number of the analyte molecules in that supramolecular complex. Thus, the total number of analyte molecules in the sample may be calculated. This method may be used with other kind of assays, including traditional well plate assays.

Pores 13 in the filter may be fabricated to have almost any size that is desired. For practical purposes size should be between 100 nm, and 5 μm. In a given filter well plate the pores should be as equal as possible. Reporter beads should preferably be 80% or less than the pore size, and more advantageously less than 50% of the pore size, and they can be only 10% of the pore size, especially, if the pore size is highly variable. This helps to minimize the nonspecific binding. The capture beads should be at least 10% bigger than the pores, and more advantageously 100% bigger. These limits are rough estimates, and actual values depend on the standard deviation of the pore size. If the size of the beads deviate more than 3 times the standard deviation from the mean pore size, the accuracy of the filtration could be 99.9%. 4.4 times the standard deviation gives the accuracy of 99.999%. This is good enough for most assays, and other errors are more meaningful. However, the pore size is highly variable in currently available commercial filters, and experimentation is needed to find out best capture and reporter bead size.

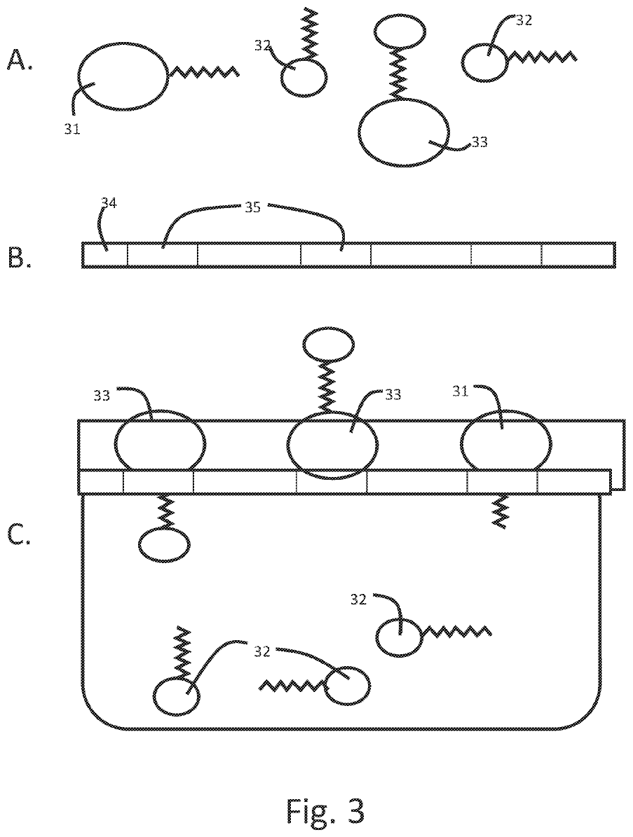

Schematics of the filtration process is depicted in FIG. 3. The assay mixture contains capture beads 31, reporter beads 32, and double bead complexes 33 (this is a shorthand notation of a double bead complex, more detailed schematics is in FIG. 2B). These beads are at this point dispersed in a water based buffer that possibly also contains water miscible solvent. Bottom of a filter plate well 34 is depicted in FIG. 3 B. Only three pores 35 are shown. FIG. 3 C depicts an ideal situation after the filtration has been completed. All capture beads are retained. They may not be bound with reporter beads 31, or be double bead complexes 33. All free reporter beads 32 are in the filtrate.

As FIG. 3. C indicates that the reporter beads may be above or below (also at a side) of the capture bead. In order of the optical detection to work in both cases, the capture bead should be as transparent as possible. Many polymers fulfil this requirement, including polystyrene, polyacrylates, polycarbonates, polyethylene, and polypropylene. Polystyrene beads are currently commonly used, and are preferred in the present invention, especially because they can be carboxyl functionalized. Preferred size of the capture beads is currently between 2 μm and 5 μm.

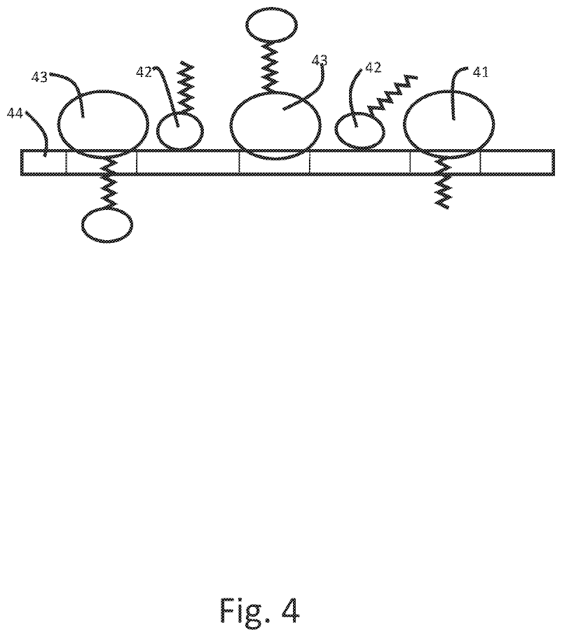

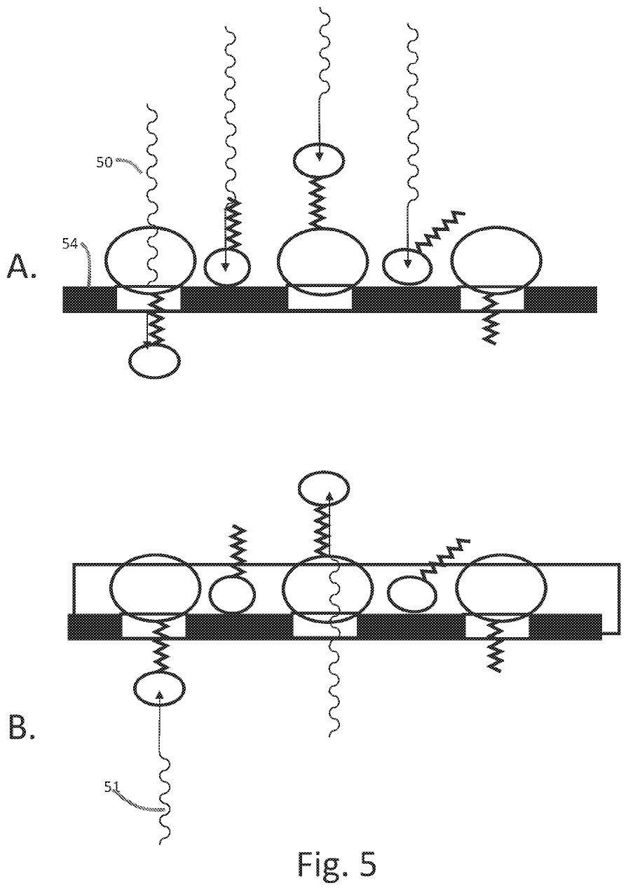

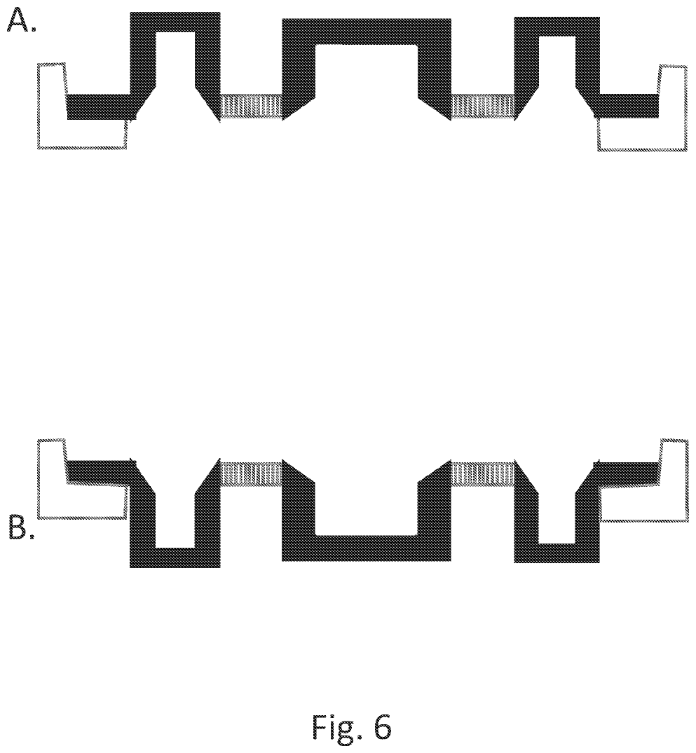

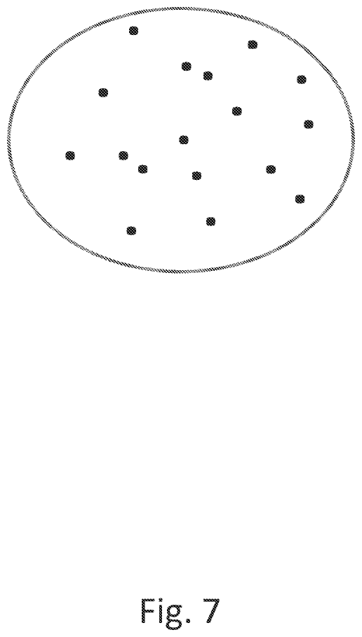

Reporter beads 42 may settle on the surface of the filter between pores during the filtration (FIG. 4). This may give false positive signal. This can be largely avoided, if the filter is not transparent, but is, for example, black (FIG. 5). In this case reading is done from the bottom side of the filter plate. Some commercial readers may be designed to read plates from below. For the readers that read results from above the filter plate may be turned upside down (FIG. 6 B). This may not be possible using commercially available filter platers, and readers, but new plates may be designed, which allow this kind of turning. These plates have the observable surface at the same height after turning them upside down (FIG. 6). This is important for the maintaining the accuracy of the optical reading. This is another enabling aspect of the present invention.

The discussion has focused on polynucleotide assays. This method is equally applicable to immunoassays. Instead of the oligonucleotide probes, antibodies will be conjugated with the beads. Presence of a certain antigen is necessary for the formation of a double bead complex.

### Instrument and Filter Well Plates

Highly efficient, and accurate well plate readers are commercially available. Some features are preferred for the purposes of the present invention. First, fluorescent detection is most advantageous. Excitation with laser enables well defined wavelength, and localized reading pixel by pixel. However, LED and other light sources may be used. We have found that time resolved fluorescence is very efficient for eliminating the nonspecific background. Only the fluorescence during first 80 microseconds should be measured. The time window may be shorter. Digital photography allows very good and fast localized detection of multiple beads with different colors. Digital photography also provides information of each pixel that is advantageous for the purposes of this invention, but not mandatory. Pixelated information gives the possibility of counting individual molecules, when using multi bead method, because each multi bead complex contains at least one analyte molecule. Ideally, this method allows the detection of one molecule, and provides an ultimate sensitivity, and accuracy. The detection limit is limited mainly by the background, i.e., by nonspecific binding. If the supramolecular complex contains two or more analyte molecules, the light intensity is correspondingly stepwise bigger, and indicates the number of analyte molecules in that pixel area. Some well plate readers measure only the total light from each well. Excitation may come from above or below. Detection is preferably done so that the excitation beam doesn't enter the detector. This is best achieved, if measurement is done perpendicular or at sharp angle to the excitation beam 50 from above (FIG. 5A), or 51 from below (FIG. 5 B). Even so that the light source, and detector are on the same side of the well plate, either above or below. Detector should preferably be below the well plate, because the signal of nonspecifically retained reporter beads may be blocked by using black filter plate (FIGS. 4, 5, and 6)). Same result may be obtained by reading above, if the filter plate is turned upside down. In order the optics to work well, the filter plate should be designed so that after turning the detection plane should be at the same level as before the turning (FIG. 6).

If the filter plate depicted in FIG. 5 is read from above, four reporter beads would be detected. If the plate is turned upside down, or the plate is read from below, only two specifically bound beads would be detected, and the correct result would be obtained. It is possible that some specifically bound reporter beads might be missed, if reading is done from the bottom side. This reduces slightly the sensitivity, but this is small price to pay for making the assay more reliable, i.e., giving less false positives. Washing in an ultrasonic bath may be used to remove non-specifically bound reporter beads. Ultrasound washing might detach also the capture beads from the upper surface. This can be prevented, if the filter plate is functionalized with biotin, and the capture beads are functionalized correspondingly with avidin, or vice versa.

Filter well plates are currently commercially available. For the purposes of this invention, it would be preferable, if all pores are essentially same size, and form a regular pattern. Regular pattern increases reliability and speed of the optical reading, because only certain spots need to be measured, and counted. This kind of regular pattern may be created by laser drilling of holes, or by a mold, or a stamp. The regular pattern may be, for example, square, or trigonal lattice.

Good quality filter membranes are currently available. A plastic plate having bottomless wells can be easily made. Filter membrane may be glued to cover the open bottoms.

### Performing the Assay

The sample can be any bodily fluid, such as blood, plasma, urine, or saliva. A small amount of the sample, for example 100 μl is placed into a collection tube that may already contain the capture, and reporter beads, and a buffer. The tube may also contain solvents, and/or enzymes and other molecules that help break down the pathogen. Solvents may include ethyl, or propyl alcohol, methyl (MFA), and dimethyl formamide (DMF), dimethyl sulfoxide. Enzymes may be proteases for genetic assays, but not for immunoassays, unless proteases are highly specific, and don't degrade the antigen, and antibodies in an undesired way. Other molecules may include detergents, such as sodium dodecyl sulphate, or buffers, such as trisHCl, or disodium phosphate. Thus, the incubation, and formation of the double bead complex may start immediately, and will be completed, when the sample reaches a laboratory provided that the sample is transported in proper temperature that is close to a room temperature. Volume of the sample may be about 500 μl at this point.

10 μl-100 μl of sample solution is pipetted into a 96-well plate. Same sample may be pipetted into multiple wells, if parallel assays are run. Preferably, all wells have a sample before the filtration step. Filtration is best done in a vacuum filtration platform, and will last only few seconds. Each sample may be washed with a buffer so that free reporter beads will be removed effectively (FIG. 3 C). Washing may be done in an ultrasonic bath so that the non-specifically bound reporter beads may be removed effectively.

The well plate is placed in a well plate reader. Many of those instruments are able to measure all 96 wells in about 5 seconds, and the results are stored in a computer. Those readers may change the well plates automatically, and may read almost 1000 well plates in one hour. Thus, in ten hour period million assays may be done with one instrument.

However, the ultimate speed is not most important aspect. Accuracy, and sensitivity are also very important as well as the cost per assay. This method is very economical, and might be the most economical assay. Actually, taking and transporting the sample might cost more than the assay itself.

### Alternative Separation Methods

The main point is to separate the reporter beads from the double or multibead complex so that the free reporter beads don't interfere with the detection of bound reporter beads. Filtration is very fast and economical method. However, alternative methods are possible, and within the scope of this invention, such as electrophoresis. Reporter beads may be coated with negative or positive functional groups, such as carboxylate, sulfonate, or amino, and trimethyl amino groups. Capture beads may be coated with similar groups. Both should preferably have either positive or negative surface charge. If they have different kind of surface charge, they will attract, and bind each other electrostatically, and are very difficult to separate. Both kind of beads tend to have same kind of surface charge anyway, because both are coated with the same kind of binding molecules, such as oligonucleotides, which are negatively charged.

Reporter beads may be much smaller than the capture beads. The radius of the capture beads may be ten times bigger than that of the reporter beads. Thus, reporter beads move much faster in water than the capture beads under equal force. The electric force may be made almost equal by using lower density of the probe oligonucleotides on the capture beads.

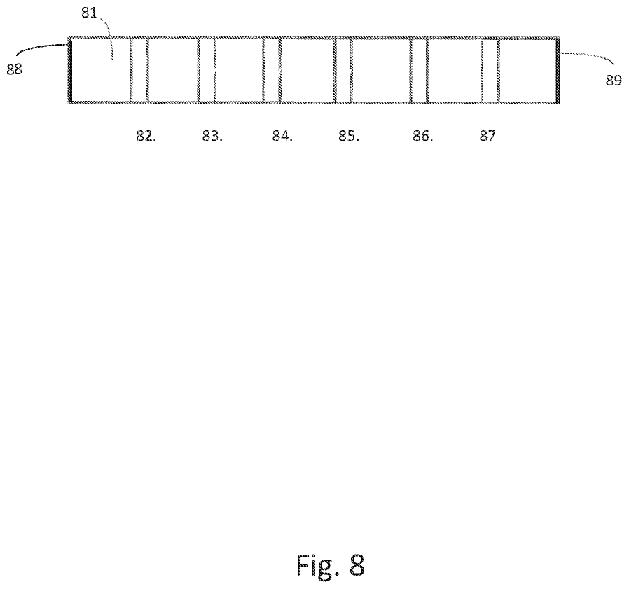

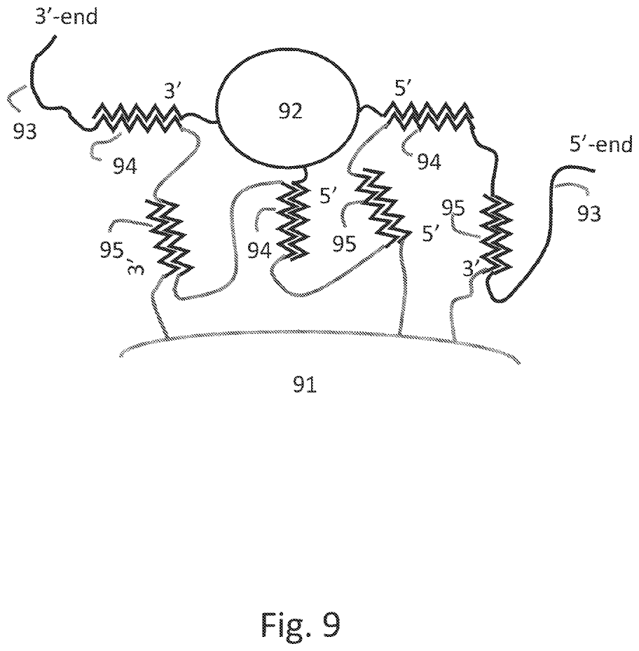

Electrophoresis may be conducted in a capillary, but this limits the sample size. Various types of chambers allow a bigger sample. The electrodes 88, and 89 may be in the opposite end of the chamber. The electrophoresis chamber may be divided with porous filters described above (FIG. 8). These have the added advantage that they reduce mixing that would otherwise impede separation of the beads, and their complexes. Filter 82 may have biggest pore size, while pore size gradually decreases so that filter 87 has the smallest pore size. Number of filters may be one, or any number that is economically feasible. In this method the capture beads remain in the volume 81, where they are originally placed, while the free reporter beads are removed. The detection may be performed visually under appropriate light source, or by digital camera. The digital camera may be in a smartphone that is supported by a suitable structure above the testing chamber. The smartphone has advantageously an app that analyzes the digital image, and reports the result to the user, and optionally to the chosen doctor's office. All parts of the chamber may be analyzed in order to ensure that the assay has been properly conducted.

In an obvious variation of the present invention the capture bead may be so large, for example, a surface of a well plate that no filtration is needed, just washing.

Without intending to limit the scope of the present invention the following examples describe the methods for the fabrication of the capture, and reporter beads, and their use.

### Example 1

Carboxylated 2 μm polystyrene beads (Spherotech Inc.) were conjugated with two oligonucleotide probes (Table 1) using a standard protocol. Shortly, 1 nmol of two oligonucleotides were added into the polystyrene bead suspension in 100 μl MES (2-[N-morpholino]ethanesulfonic acid) buffer. 0.3 mg of EDC was added, and mixed with Vortex mixer. After 15 min another 0.3 mg of EDC (1-ethyl-3 (-3dimethylaminopropyl) carbodiimide hydrochloride) was added. After 30 min the mixture was centrifugated at 10 k rpm 5 min. Supernatant was removed, and the beads were washed with water/DMF 3:1 mixture, and centrifugated again.

Reporter beads were 0.6 μm polyimide beads containing europium chelate. They were conjugated similarly with three oligonucleotide probes (Table 1).

TABLE 1

| | |
|---|---|
| Capture probe 1 | GCCAATGCGCGACATTCCGA |
| | (SEQ ID NO. 1) |
| Capture probe 2 | AGAACGCTGAAGCGCTGGGG |
| | (SEQ ID NO. 2) |
| Reporter probe 1 | TACACTCGATCGTACTCCGC |
| | (SEQ ID NO. 3) |
| Reporter probe 2 | CGTGGCCTCGGTGAAAATGT |
| | (SEQ ID NO. 4) |
| Reporter probe 3 | CTCCCTAGCATTGTTCACTG |
| | (SEQ ID NO. 5) |
| Target | Synthetic mixture of SARS-COV-2 |
| | RNA fragments (Sigma Aldrich) |

### Example 2

10\hat{ }5 capture beads and 10\hat{ }4 reporter beads were dispersed into 100 μl of 1 pmol of synthetic fragments of SARS-COV-2 in a hybridization buffer (Mullis, et al.), and incubated 30 min. 50 μl of the mixture was filtered through one well in a 96-well filter plate. Filter well plate was self-made by removing the bottoms of a commercial well plate, and gluing a filter paper having 5 μm pores on the bottom holes. The wells ware inspected and photographed using a microscope, and an ultraviolet light source. The image is in FIG. 7. The image was processed so that the red spots were turned black, and the black background was turned white.

1. A method for the detection of an analyte in a sample by
a. adding capture beads, and
b. reporter beads, and
c. incubating the mixture, and
d. filtering the mixture through a filter that has pore size that is at least 100% bigger than the diameter of the reporter beads, but at least 20% smaller than the diameter of the capture beads, and
e. using an optical reader to measure the amount of the reporter beads retained by the filter.

2. The method of claim 1, in which the analyte is an oligonucleotide, and the capture, and reporter beads are conjugated with complementary oligonucleotide probes.

3. The method of claim 1, in which the analyte is an antigen, and the capture, and reporter beads are conjugated with antibodies that are specific for the said antigen.

4. The method of claim 1, in which the optical reader measures fluorescent light.

5. The method of claim 4, in which the optical reader is a digital camera, and the color information if provided for each pixel.

6. The method of claim 5, in which the digital information is used to calculate the number of analyte molecules.

7. The method of claim 1, in which two or more different reporter beads are used to detect the same analyte so that several different colors are detected in a deliberately chosen ratio, when an analyte is present.

8. The method of claim 1, in which the said filter is a filter well plate.

9. The method of claim 8, in which the said filter well plate is black.

10. The method of claim 8, in which at least part of the filter plate is functionalized with biotin, or avidin.

11. The method of claim 8, in which the bottom side of the said filter well plate is washed in an ultrasonic bath before the optical measurement.

12. The method of claim 8, in which the bottom side of the said filter plate is optically measured.
